(19) United States
(12) Patent Application Publication
(10) Pub. No.: US 2010/0318104 A1
(43) Pub. Date: Dec. 16, 2010

(54) Surgical forceps for applying an aneurysm clip

(76) Inventor: Daniel LAZIC, Tuttlingen (DE)

Correspondence Address: WALTER A. HACKLER, 2372 S.E. BRISTOL, SUITE B, NEWPORT BEACH, CA, 92660-0755, US

(21) Appl. No.: 12/813,015

(22) Filed: Jun. 10, 2010

(30) Foreign Application Priority Data
Jun. 15, 2009 (DE) 10 2009 026 928.2

(51) Int. Cl. A61B 17/10

(52) U.S. Cl. 606/142

(57) ABSTRACT

In a surgical forceps comprising two rotatably connected forceps parts which are preloaded into a first forceps end position, a locking arm, one end of which is mounted to the first forceps part and the other end of which is slidably guided on the second forceps part, and a locking element which is mounted to the second forceps part and is disposed in the path of movement of the guided end of the locking arm, wherein in a intermediate position of the forceps, the locking element is engaged behind by the guided end of the locking arm and is thereby locked in the intermediate position of the forceps against the preload, in accordance with the invention the locking element is movable out of its locked position disposed in the path of movement of the guided end of the locking arm to a release position in which the locking element is not disposed in the path of movement of the guided end of the locking arm.

### CROSS REFERENCE TO RELATED APPLICATIONS

This application claims priority under 35 U.S.C. §119(a) to German Patent Application No. 10 2009 026 928.2, filed Jun. 15, 2009, the entire contents of which are hereby incorporated by reference.

### TECHNICAL HELD

The invention relates to surgical forceps or the like, in particular, clip applying forceps, comprising two rotatably connected forceps parts which are preloaded into a first forceps end position, a locking arm, one end of which is mounted to the first forceps part and the other end of which is slidably guided on the second forceps part, and a locking element which is mounted to the second forceps part and is disposed in the path of movement of the guided end of the locking arm, wherein in one intermediate position of the forceps, the locking element is engaged behind by the guided end of the locking arm and is thereby locked in the intermediate position of the forceps against the preload.

### BACKGROUND

Clip applying forceps of this type are e.g. disclosed by DE 31 03 352 A1.

These conventional clip applying forceps comprise two rotatably connected forceps parts which can be pivoted with respect to each other and the front ends of which are formed like pincers for receiving a clip. At the rear end, the two forceps parts are formed like spring arms which are connected to each other and preload the clip applying forceps into the open forceps end position. A leaf spring is mounted to the first forceps part to act as a locking arm, the other end of which is slidably guided on a sliding guide of the second forceps part. The sliding guide has several steps, which are engaged behind by the free end of the locking arm in respective intermediate positions of the forceps when the two forceps parts are pressed together, thereby locking the two forceps parts with respect to each other in the intermediate positions of the forceps against the preload. Complete opening of the forceps is only possible when the guided end of the locking arm has previously been moved out of the effective range of the sliding guide by fully pressing the forceps parts together.

In contrast thereto, it is the object of the present invention to operate forceps of the above-mentioned type with the lockable intermediate position of the forceps as well as without the lockable intermediate position of the forceps.

### SUMMARY

This object is achieved in accordance with the invention in that the locking element is movable out of its locking position disposed in the path of movement of the guided end of the locking arm into a release position in which the locking element is not disposed in the path of movement of the guided end of the locking arm.

In accordance with the invention, the locking element can be switched between the locking position and the release position. The user can therefore easily change between the two positions without any structural alterations. In accordance with the invention, this switchable locking element can be used for surgical forceps, such as e.g. clip applying forceps, or for other surgical instruments having two branches which can be rotated relative to each other such as e.g. needle holders, scissors, vessel clips, tweezers etc.

With particular preference, the locking element is disposed on the second forceps part such that it can be rotated between its locking position and its release position. This embodiment represents a simple low-wear construction which ensures user-friendly operation of the locking element.

The locking element is advantageously disposed on the second forceps part in such a fashion that it can be adjusted in the guiding direction of the locking arm in order to enable individual adjustment of the intermediate position of the forceps.

With particular preference, the guided end of the locking arm is bent to form a hook shape or U-shape, with which the locking arm engages behind the locking element in the intermediate position of the forceps.

Further advantages of the invention can be extracted from the description, the claims and the drawing. The features mentioned above and below may also be used individually or collectively in arbitrary combination. The embodiments shown and described are not to be understood as exhaustive enumeration but have exemplary character for describing the invention.

### BRIEF DESCRIPTION OF DRAWINGS

FIG. 1 shows a perspective view of the inventive clip applying forceps;

FIG. 2 shows a detailed view of a locking unit shown in FIG. 1 for locking the forceps in a certain intermediate position of the forceps by means of a rotatably disposed locking element which is shown in the locking position;

FIG. 3 shows a bottom view of the locking unit shown in FIG. 2; and

FIG. 4 shows the locking unit with the locking element in the release position in a detailed view analogously to FIG. 2

### DETAILED DESCRIPTION

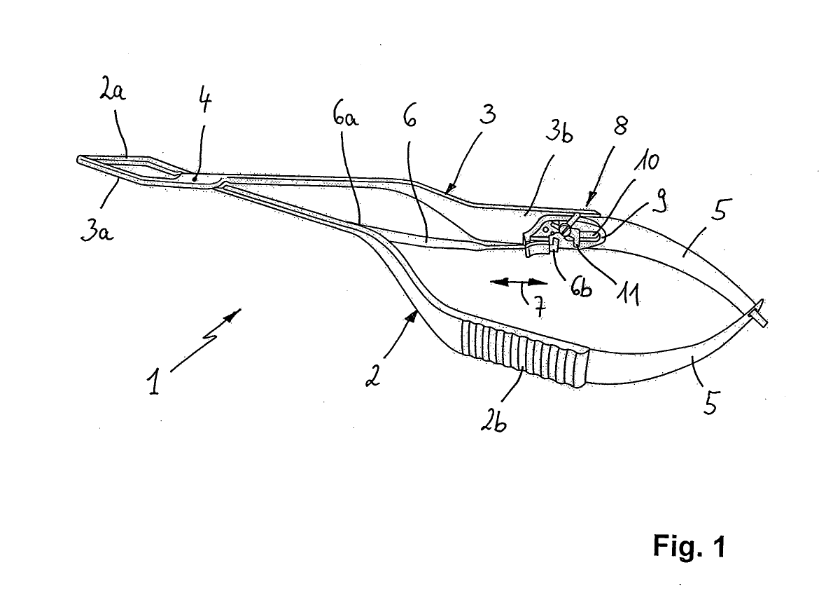

The aneurysm clip applying forceps 1 shown in FIG. 1 comprise two forceps parts (branches) 2, 3, which are rotatably connected to each other at 4 and each have a shorter front gripping arm 2a, 3a and a longer rear operating arm 2b, 3b. The gripping arms 2a, 3a are designed to receive an aneurysm clip (not shown). The operating arms 2b, 3b have leaf springs 5 at the rear ends, which are inserted into each other at their free ends, thereby preloading the forceps 1 into the forceps end position shown in FIG. 1 with closed front gripping arms 2a, 3a and open operating arms 2b, 3b. When the two operating arms 2b, 3b are pressed together starting from this closed forceps end position against the restoring force of the leaf springs 5, the gripping arms 2a, 3a are opened.

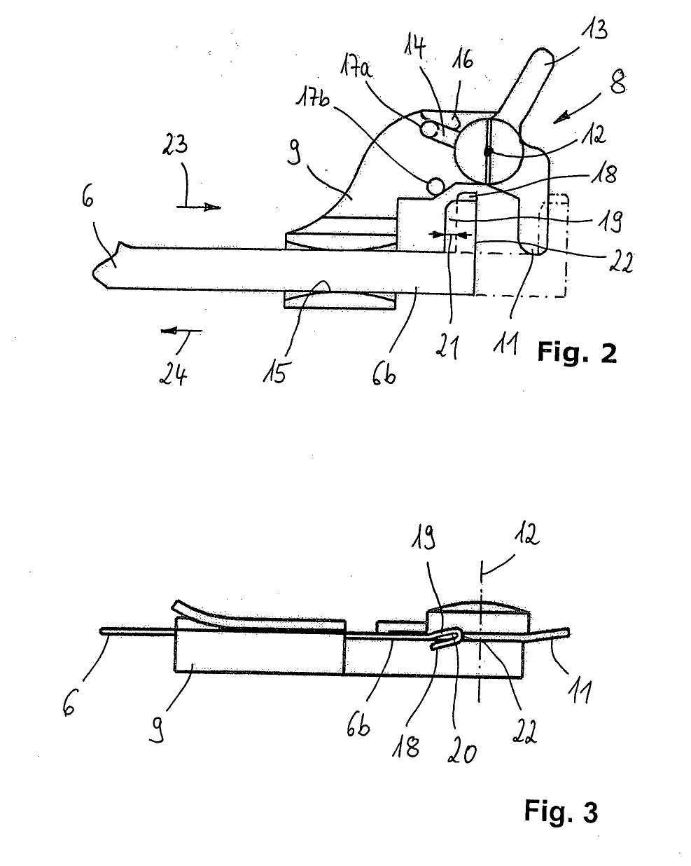

The forceps 1 further comprise a locking arm 6, one end 6a of which is mounted to the first forceps part 2 and the other end 6b of which is displaceably guided (double arrow 7) on the second forceps part 3, and a locking unit 8 mounted to the second forceps part 3 for locking the forceps 1 in a certain opened intermediate position of the forceps. The locking unit 8 comprises a base plate 9 which can be adjusted via an elongated hole 10 on the second forceps part 3 in the guiding direction 7 and can be locked in the desired position by means of a screw (not shown). A locking element 11 formed as a locking arm in the present case is disposed on the base plate 9 between a locking position and a release position such that it can be rotated about the axis of rotation 12 (FIG. 2). The locking arm 6 is guided on the base plate 9 such that it can be displaced in the direction of the double arrow 7.

As is shown in FIG. 2, the locking element 11 has an operating handle 13 and a catch arm 14. In addition to the guidance 15 for the locking arm 6, the base plate 9 is also provided with a rotary stop 16 which acts in both directions, and two catch recesses 17a, 17b. The free end 6b of the locking arm 6 widens in an upward direction at its end, as is shown in FIG. 3, and is bent in a U-shape in an inward direction, i.e. towards the second forceps part 3, in order to thereby form a receptacle 20 between the bent end section 18 and the non-bent end section 19 of the locking arm 6, which receptacle 20 is open towards the axis of rotation 4.

Based on the bottom of the receptacle 20, the bent end section 18 is shorter than the non-bent end section 19 by the measure 21.

FIGS. 1 to 3 show the locking element 11 in its locking position and the locking arm 6 in its initial position when the forceps 1 are closed. The U-shaped bent end 22 of the locking element 11 engages in the path of movement of the locking arm 6 and is secured in this locking position by the catch arm 14 which abuts one side of the rotary stop 16 and is locked into the catch recess 17a.

When the two operating arms 2b, 3b are pressed together against the restoring force of the leaf springs 5 for opening the forceps 1, the U-shaped bent end 22 of the locking arm 6 is displaced in the direction 23, i.e. away from the axis of rotation 4, past the locking element 11 into the position shown in FIG. 2 with dash-dotted lines, in which the longer end section 19 laterally abuts the locking element 11. To be more precise, the shorter end section 18 of the locking arm 6 thereby initially runs up the locking element 11, thereby deflecting and preloading the U-shaped bent end 22 laterally towards the first operating arm 2b, and when the shorter end section 18 has been pushed past the locking element 11, it is pivoted back towards the second operating arm 2b and in abutment with the longer end section 19 and is still preloaded. The running up of the shorter end section 18 is supported by the position of the U-shaped bent end 22 which is slightly inclined with respect to the guiding direction 7 (FIG. 3).

When the two operating arms 2b, 3b are no longer pressed together and are consequently opened by the restoring force of the leaf springs 5, the U-shaped bent end 22 of the locking arm 5 is displaced in the opposite direction 24, i.e. in the direction towards the axis of rotation 4, The receptacle 20 is thereby pushed onto the locking element 11 until the locking arm 6 finally abuts the locking element 11 with its U-shaped bent end 22, thereby locking the two operating arms 2b, 3b with respect to each other against the restoring force of the leaf springs 5. In other words, the locking element 11 is engaged behind by the U-shaped bent end 22 of the locking arm 6 in the direction 24, thereby locking the forceps 1 in the intermediate position of the forceps with partially opened gripping arms 2a, 3a. The intermediate position of the forceps can be adjusted by displacing the locking unit 8 along the elongated hole 10.

For releasing the locking, the two operating arms 2b, 3b are initially pressed together until the U-shaped bent end 22 of the locking arm 6 has been completely pushed past the locking element 11 in the direction 23 and can pivot back in the direction towards the second operating arm 2b. When the two operating arms 2b, 3b are no longer pressed together and are consequently opened by the restoring force of the leaf springs 5, the locking arm 6 runs with its longer end section 19 up the locking element 11. The U-shaped bent end 22 is thereby laterally deflected in the direction towards the second operating arm 3b due to the still acting preload, and is thereby guided on the other side of the locking element 11 in the direction 24 past the locking element 11 until the closed forceps end position shown in FIG. 1 is reached.

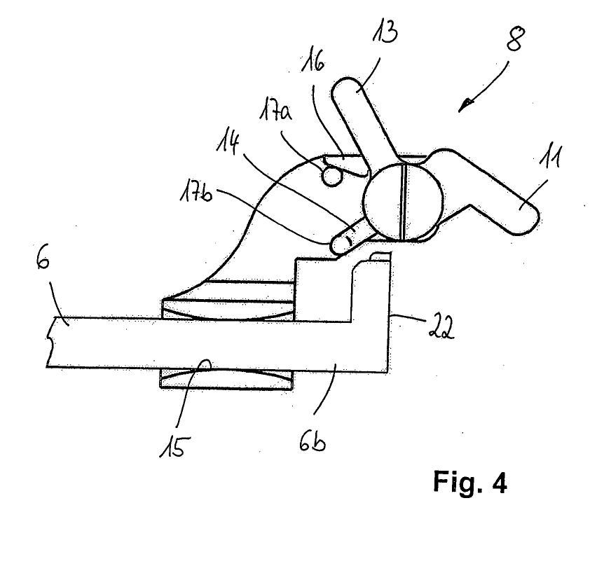

In FIG. 4, the locking element 11 is rotated into its release position by means of the operating handle 13, in which position the locking element 11 does not engage in the path of movement of the locking arm 6. In this release position, the locking element 11 is secured by the locking arm 14 which abuts the other side of the rotary stop 16 and is locked into the other catch recess 17b. When the two operating arms 2b, 3b, are correspondingly further pressed together, the forceps 1 can be individually opened or closed without locking.

The above-described locking is not limited to forceps but can also be applied for other surgical instruments having two branches which can be rotated with respect to one another, such as e.g. needle holders, scissors, vessel clips, tweezers, etc.

What is claimed is:

1. Clip applying surgical forceps comprising:
two rotatably connected forceps parts preloaded into a first forceps end position;
a locking arm, one end mounted to the first forceps part and another end slidably guided on the second forceps part; and
a locking element mounted to the second forceps part and disposed in a movement path of the locking arm guided end, wherein in an intermediate position of the forceps, the locking element is engaged by the locking arm guided end and is thereby locked in the intermediate position of the forceps against the preload, and
wherein the locking element is movable out of engagement to a release position in which the locking element is not disposed in the movement path of the locking arm guided end.

2. Forceps according to claim 1, wherein the locking element is disposed on the second forceps part such that it can be rotated between its locked position and its release position.

3. Forceps according to claim 1, wherein the locking element is disposed on the second forceps part such that it can be adjusted in the guiding direction of the locking arm.

4. Forceps according to claim 1, wherein the locking arm has a hook-shaped or U-shaped bent end at its guided end, by means of which the locking arm engages behind the locking element in the intermediate position of the forceps.

5. Forceps according to claim 1, wherein, when the two forceps parts are moved in the direction towards the other second forceps end position, the guided end of the locking arm is laterally deflected by the locking element and is thereby preloaded in the direction towards its position engaging behind the locking element.

6. Forceps according to claim 1, wherein the locking element is clamped or locked in its locking position and/or release position.

7. Forceps according to claim 1, wherein at least one rotary stop is provided, on which the locking element abuts in its locking position and/or release position.

8. Forceps according to claim 1, wherein the locking element has a handle for moving between the locking position and the release position.

9. Forceps according to any one of the preceding claims, wherein the rear operating arms of the two forceps parts have leaf springs which are connected to each other at their free ends for generating the preload.

10. Forceps according to claim 1, wherein the locking element is mounted to the second forceps part by means of a base plate.

11. Forceps according to claim 10, wherein the locking arm is guided on the base plate such that it can be displaced in the guiding direction.

12. Forceps according to claim 2, wherein the locking element is disposed on the second forceps part such that it can be adjusted in the guiding direction of the locking arm, wherein the locking arm has a hook-shaped or U-shaped bent end at its guided end, by means of which the locking arm engages behind the locking element in the intermediate position of the forceps, wherein, when the two forceps parts are moved in the direction towards the other second forceps end position, the guided end of the locking arm is laterally deflected by the locking element and is thereby preloaded in the direction towards its position engaging behind the locking element, wherein the locking element is clamped or locked in its locking position and/or release position, wherein at least one rotary stop is provided, on which the locking element abuts in its locking position and/or release position, wherein the locking element has a handle for moving between the locking position and the release position, wherein the rear operating arms of the two forceps parts have leaf springs which are connected to each other at their free ends for generating the preload, and wherein the locking element is mounted to the second forceps part by means of a base plate.

13. Forceps according to claim 12, wherein the locking arm is guided on the base plate such that it can be displaced in the guiding direction.
